(19) United States
(12) Patent Application Publication
(10) Pub. No.: US 2021/0177485 A1
(43) Pub. Date: Jun. 17, 2021

(54) MEDICAL HEATER, TREATMENT INSTRUMENT, AND PRODUCTION METHOD FOR TREATMENT INSTRUMENT

(71) Applicant: OLYMPUS CORPORATION, Tokyo (JP)

(72) Inventor: Tsunetaka AKAGANE, Tokyo (JP)

(73) Assignee: OLYMPUS CORPORATION, Tokyo (JP)

(21) Appl. No.: 17/183,557

(22) Filed: Feb. 24, 2021

Related U.S. Application Data
(63) Continuation of application No. PCT/JP2018/032475, filed on Aug. 31, 2018.

(51) Int. Cl. A61B 18/08

(52) U.S. Cl. CPC A61B 18/08; A61B 2018/00148

(57) ABSTRACT

A medical heater includes: a heat generating portion that is made of a material containing nickel, the heat generating portion being configured to generate heat when energized; and a passivation film that is made of nickel fluoride, the passivation film being configured to cover at least a part of a surface of the heat generating portion.

### CROSS-REFERENCE TO RELATED APPLICATION

This application is a continuation of International Application No. PCT/JP2018/032475, filed on Aug. 31, 2018, the entire contents of which are incorporated herein by reference.

### BACKGROUND

### 1. Technical Field

The present disclosure relates to a medical heater, a treatment tool, and a method of manufacturing a treatment tool.

### 2. Related Art

In the related art, there is known a treatment tool that applies energy to a site to be treated (hereinafter referred to as a target site) in a living tissue to treat the target site (see, for example, JP 2005-348820 A).

The treatment tool described in JP 2005-348820 A includes first and second gripping members that grip the target site. One of the first and second gripping members is provided with a medical heater having a heat generating portion that generates heat when energized, and a treatment member that comes into contact with the target site when the target site is gripped by the pair of gripping members. Further, the heat from the medical heater is transferred to the target site gripped by the first and second gripping members via the treatment member in the treatment tool. As a result, the target site is treated.

### SUMMARY

In some embodiments, a medical heater includes: a heat generating portion that is made of a material containing nickel, the heat generating portion being configured to generate heat when energized; and a passivation film that is made of nickel fluoride, the passivation film being configured to cover at least a part of a surface of the heat generating portion.

In some embodiments, a treatment tool includes: a treatment member having a treatment surface for treating a living tissue and an installation surface forming front and back surfaces of the treatment member with the treatment surface; and a medical heater configured to heat the treatment member. The medical heater includes: a substrate that is made of a material having electrical insulation and has a first plate surface and a second plate surface forming front and back surfaces of the substrate; a heat generating portion that is made of a material containing nickel and generates heat when energized; a passivation film that is made of nickel fluoride, the passivation film being configured to cover at least a part of a surface of the heat generating portion; a first connection portion and a second connection portion to which wiring members are electrically connected, respectively; and an electric path portion that serves as an energization path to the heat generating portion, the heat generating portion, the first connection portion, the second connection portion, and the electric path portion are provided on the first plate surface in a state of being connected in series along a longitudinal direction of the substrate in an order of the first connection portion, the heat generating portion, the electric path portion, and the second connection portion, the heat generating portion has a higher resistance value than the first connection portion, the second connection portion, and the electric path portion, and the substrate is made of a flexible material, is folded with a folding line orthogonal to the longitudinal direction of the substrate as a reference in a state where the first plate surface forms an outer surface of the medical heater, and is installed in a state where the heat generating portion faces the installation surface.

In some embodiments, provided is a method of manufacturing a treatment tool. The method includes: forming a heat generating portion, which is made of a material containing nickel and generates heat when energized, a first connection portion and a second connection portion to which wiring members are electrically connected, respectively, and an electric path portion that serves as an energization path to the heat generating portion on a first plate surface of a substrate in a state where the first connection portion, the heat generating portion, the electric path portion, and the second connection portion are sequentially connected in series along a longitudinal direction of the substrate; and performing surface modification on at least a part of a surface of the heat generating portion in an atmosphere of a gas containing fluorine to form a passivation film made of nickel fluoride on at least the part of the surface of the heat generating portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating a treatment system according to a an exemplary embodiment;

FIG. 2 is a view illustrating a gripping portion;

FIG. 3 is a view illustrating a gripping portion;

FIG. 4 is a view illustrating a medical heater;

FIG. 5 is a view illustrating the medical heater;

FIG. 6 is a flowchart illustrating a method of manufacturing a treatment tool;

FIG. 7 is a view illustrating the method of manufacturing the treatment tool; and

FIG. 8 is a view illustrating a medical heater according to another exemplary embodiment.

### DETAILED DESCRIPTION

Hereinafter, modes (hereinafter, embodiments) for carrying out the disclosure will be described with reference to the drawings. Incidentally, the disclosure is not limited to the embodiments to be described below. Further, the same parts are denoted by the same reference signs when the drawings are described.

### Schematic Configuration of Treatment System

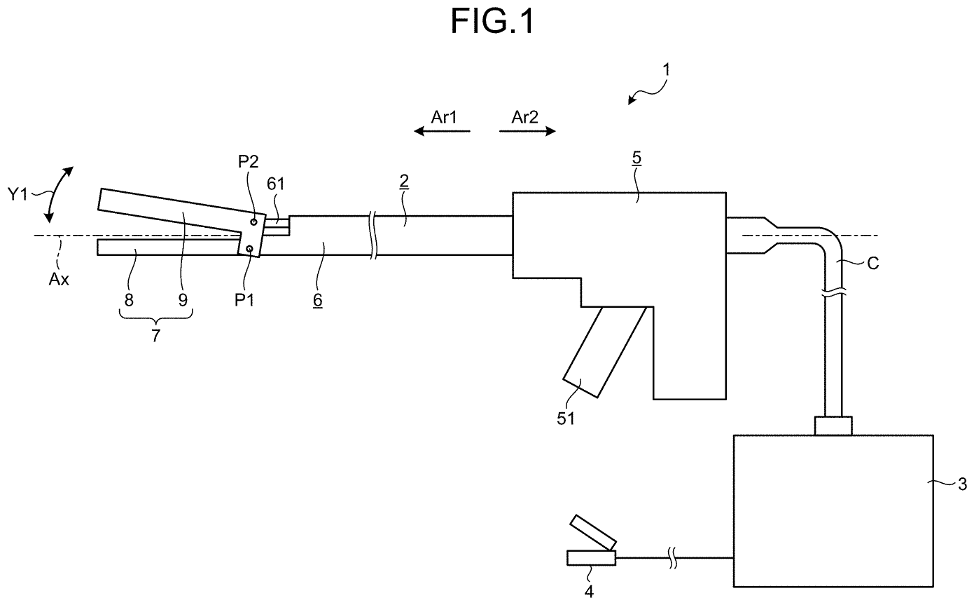

FIG. 1 is a view illustrating a treatment system 1 according to an exemplary embodiment.

The treatment system 1 applies thermal energy to a site to be treated (hereinafter referred to as a target site) in a living tissue to treat the target site. Here, the treatment means, for example, coagulation and incision of the target site. As illustrated in FIG. 1, the treatment system 1 includes a treatment tool 2, a control apparatus 3, and a foot switch 4.

Configuration of Treatment Tool

The treatment tool 2 is, for example, a surgical treatment tool configured to treat a target site in the state of passing through an abdominal wall. As illustrated in FIG. 1, the treatment tool 2 includes a handle 5, a shaft 6, and a gripping portion 7.

The handle 5 is a part held by an operator. Further, the handle 5 is provided with an operation knob 51 as illustrated in FIG. 1.

The shaft 6 has a substantially cylindrical shape. Incidentally, one side of the shaft 6 along a central axis Ax will be referred to as a distal end side Ar1 (FIG. 1), and the other side will be referred to as a proximal end side Ar2 (FIG. 1), hereinafter. Further, the shaft 6 is attached to the handle 5 by inserting a part of the proximal end side Ar2 from the distal end side Ar1 of the handle 5 into the inside of the handle 5. Further, a movable member 61 (FIG. 1), which moves back and forth along the central axis Ax in response to an operation of the operation knob 51 performed by the operator, is arranged inside the shaft 6. In addition, an electric cable C (FIG. 1) has one end connected to the control apparatus 3 and the other end arranged up to the gripping portion 7 after passing through the inside of the handle 5 and the shaft 6.

Configuration of Gripping Portion

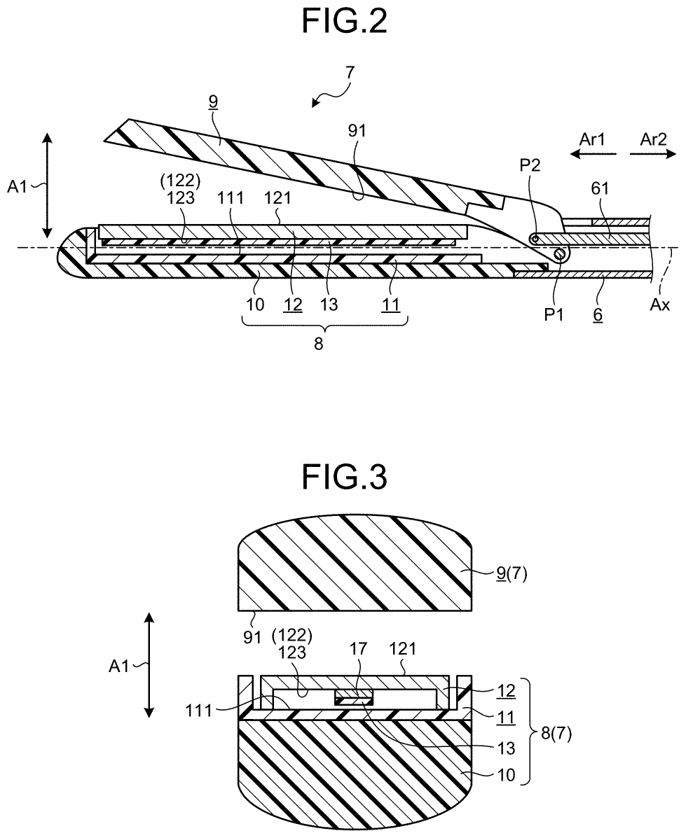

FIGS. 2 and 3 are views illustrating the gripping portion 7. Specifically, FIG. 2 is a cross-sectional view of the gripping portion 7 cut along a plane along the central axis Ax. FIG. 3 is a cross-sectional view of the gripping portion 7 cut along a plane orthogonal to the central axis Ax.

The gripping portion 7 is a portion that treats the target site in the state of gripping the target site. As illustrated in FIGS. 1 to 3, the gripping portion 7 includes first and second gripping members 8 and 9.

The first and second gripping members 8 and 9 can be open and closed in a direction of arrow Y1 (FIG. 1) according to the operation of the operation knob 51 performed by the operator.

Configuration of First Gripping Member

The first gripping member 8 is arranged on the lower side in FIGS. 2 and 3 with respect to the second gripping member 9. As illustrated in FIG. 2 or 3, the first gripping member 8 includes a support member 10, a heat insulating member 11, a treatment member 12, and a medical heater 13.

The support member 10 has an elongated shape extending in a longitudinal direction connecting a distal end and a proximal end of the gripping portion 7 (in FIG. 2, the left-right direction (direction along the central axis Ax), and has one end fixed to an end portion on the distal end side Ar1 of the shaft 6. Further, the support member 10 supports the heat insulating member 11, the treatment member 12, and the medical heater 13 by an upper surface in FIGS. 2 and 3.

Examples of a material forming the support member 10 described above may include a metal material such as stainless steel and titanium.

The heat insulating member 11 has an elongated shape extending in the longitudinal direction of the gripping portion 7, and is fixed to the upper surface of the support member 10 in FIGS. 2 and 3.

In the heat insulating member 11, a recess 111 extending from a proximal end of the heat insulating member 11 to the distal end side Ar1 is formed on an upper surface in FIGS. 2 and 3. Further, the heat insulating member 11 supports the treatment member 12 and the medical heater 13 in the recess 111.

Examples of a material forming the heat insulating member 11 described above can include a resin material having a low thermal conductivity such as polyetheretherketone (PEEK). That is, since the heat insulating member 11 having a low thermal conductivity is arranged between the treatment member 12 and the medical heater 13, and the support member 10, the heat from the medical heater 13 can be efficiently transferred to the treatment member 12.

The treatment member 12 has an elongated shape extending in the longitudinal direction of the gripping portion 7 and is fixed in the recess 111.

An upper surface of the treatment member 12 in FIGS. 2 and 3 comes into contact with the target site in a state where the target site is gripped by the first and second gripping members 8 and 9. That is, the surface functions as a treatment surface 121 (FIGS. 2 and 3) that applies thermal energy to the target site. Incidentally, “applying thermal energy to the target site” means transferring the heat from the medical heater 13 to the target site. In an exemplary embodiment, the treatment surface 121 is formed of a flat surface orthogonal to a direction A1 (FIGS. 2 and 3) in which the first and second gripping members 8 and 9 face each other when the first and second gripping members 8 and 9 are set in the closed state of gripping the target site.

In an exemplary embodiment, the treatment surface 121 is formed of the flat surface, and may be formed of other shapes such as a convex shape and a concave shape without being limited thereto. The same applies to a gripping surface 91 to be described later.

In addition, in the treatment member 12, a recess 123 (FIGS. 2 and 3) extending from a proximal end to a distal end of the treatment member 12 is formed on an installation surface 122 forming front and back surfaces with the treatment surface 121. Further, the treatment member 12 supports the medical heater 13 by a bottom surface of the recess 123.

Examples of a material forming the treatment member 12 described above can include highly thermally conductive copper, silver, aluminum, molybdenum, tungsten, graphite, or composite materials thereof.

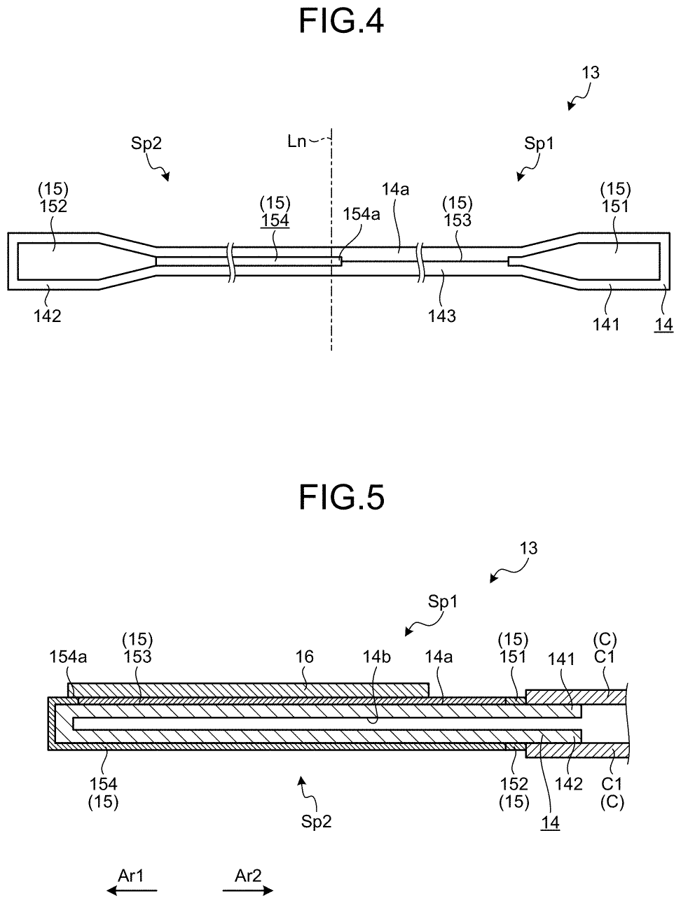

FIGS. 4 and 5 are views illustrating the medical heater 13. Specifically, FIG. 4 is a view of the medical heater 13 in a state before a substrate 14 is folded as viewed from a first plate surface 14a of the substrate 14. FIG. 5 is a cross-sectional view of the medical heater 13 in a state after the substrate 14 is folded, which is cut along a plane orthogonal to a width direction (horizontal direction in FIG. 3) of the substrate 14.

The medical heater 13 is a sheet heater that partially generates heat when energized. As illustrated in FIG. 4 or 5, the medical heater 13 includes the substrate 14, a conductive portion 15, and a passivation film 16 (FIG. 5).

The substrate 14 is a sheet-like flexible substrate made of a resin material having electrical insulation such as polyimide. The substrate 14 is formed in an elongated shape, and includes first and second wide portions 141 and 142 located at both ends in a longitudinal direction (left-right direction in FIG. 4), respectively, and a narrow portion 143 which is located between the first and second wide portions 141 and 142 and connects the first and second wide portions 141 and 142.

Here, a width dimension (length dimension in the up-down direction in FIG. 4) of the narrow portion 143 is set to be substantially uniform along the longitudinal direction. In addition, the width dimension of the narrow portion 143 is set to be smaller than those of the first and second wide portions 141 and 142.

The conductive portion 15 is formed by patterning a metal thin film deposited by vapor deposition or sputtering using photolithography on the first plate surface 14a between the first plate surface 14a (FIGS. 4 and 5) and a second plate surface 14b (FIG. 5) forming front and back surfaces of the substrate 14. As illustrated in FIG. 4 or 5, the conductive portion 15 includes first and second connection portions 151 and 152, a heat generating portion 153, and an electric path portion 154.

As illustrated in FIG. 4, the first and second connection portions 151 and 152 are provided in the first and second wide portions 141 and 142, respectively. Further, a pair of lead wires C1 (FIG. 5) constituting the electric cable C are electrically connected to the first and second connection portions 151 and 152, respectively.

The heat generating portion 153 has one end connected to the first connection portion 151, and the other end side extending toward the second connection portion 152 while meandering in a wavy shape, for example. Incidentally, the heat generating portion 153 is not limited to the shape that extends while meandering in a wavy shape, and may have a shape that extends linearly from the first connection portion 151 toward the second connection portion 152.

The electric path portion 154 is a portion that serves as an energization path to the heat generating portion 153, and has one end connected to the other end of the heat generating portion 153, and the other end side extending linearly toward the second connection portion 152. Here, the one end connected to the heat generating portion 153 in the electric path portion 154 corresponds to a heat-generating-side end portion 154a (FIGS. 4 and 5) according to the disclosure. Further, the other end of the electric path portion 154 is connected to the second connection portion 152.

That is, the first and second connection portions 151 and 152, the heat generating portion 153, and the electric path portion 154 are provided on the first plate surface 14a in the state of being connected in series in an order of the first connection portion 151, the heat generating portion 153, the electric path portion 154, and the second connection portion 152 along the longitudinal direction of the substrate 14.

Further, a resistance value of the heat generating portion 153 is set to be higher than those of the first and second connection portions 151 and 152 and the electric path portion 154 by setting each of the first and second connection portions 151 and 152, the heat generating portion 153, and the electric path portion 154 to have predetermined total length and cross-sectional area. Therefore, when a voltage is applied to the first and second connection portions 151 and 152 via the pair of lead wires C1 under the control of the control apparatus 3, the heat generating portion 153 mainly generates heat.

Examples of a material forming the conductive portion 15 described above can include a material containing nickel, specifically, stainless steel, nickel, or a nickel alloy. Incidentally, if at least the heat generating portion 153 is made of the material containing nickel, the first and second connection portions 151 and 152 and the electric path portion 154 may be made of a material different from that of the heat generating portion 153.

The passivation film 16 is made of nickel fluoride and covers a part of a surface of the conductive portion 15 as illustrated in FIG. 5. Specifically, the passivation film 16 covers a surface of the heat-generating-side end portion 154a, and further extends from the surface of the heat-generating-side end portion 154a toward the first connection portion 151 to cover a part of a surface of the heat generating portion 153.

The medical heater 13 described above is fixed to a bottom surface of the recess 123 using an adhesive sheet 17 (FIG. 3) in the state where the substrate 14 is folded.

Here, the adhesive sheet 17 is located between the bottom surface of the recess 123 and the medical heater 13, and causes the bottom surface and the medical heater 13 to adhere to each other. The adhesive sheet 17 is formed by mixing a material having a high thermal conductivity, a high temperature resistance, and adhesiveness, for example, an epoxy resin with ceramic having a high thermal conductivity such as alumina and aluminum nitride.

The substrate 14 is folded in a state where the first plate surface 14a forms an outer surface of the medical heater 13 as illustrated in FIG. 5 with a folding line Ln (FIG. 4) orthogonal to the longitudinal direction of the substrate 14 and located substantially at the center of the longitudinal direction as a reference. In other words, the substrate 14 is folded in a state where the second plate surface 14b is located on the inner side with the folding line Ln as the reference. In this state, the first and second wide portions 141 and 142 face each other. Incidentally, the folding line Ln is not limited to one that is exactly orthogonal to the longitudinal direction of the substrate 14, and also includes those crossing the longitudinal direction in a range of a predetermined angle.

Hereinafter, a region on the first connection portion 151 side with respect to the folding line Ln will be referred to as a treatment-side region Sp1, and a region on the second connection portion 152 side with respect to the folding line Ln will be referred as a back-surface-side region Sp2, for convenience of the description.

As illustrated in FIG. 4, the electric path portion 154 is provided so as to straddle the folding line Ln. Therefore, the first connection portion 151, the heat generating portion 153, and the heat-generating-side end portion 154a are located in the treatment-side region Sp1. In addition, the second connection portion 152 and a region of the electric path portion 154 other than the heat-generating-side end portion 154a are located in the back-surface-side region Sp2.

Further, the substrate 14 is folded with the folding line Ln as the reference as described above, and is fixed to the bottom surface of the recess 123 by the adhesive sheet 17 in a state where the treatment-side region Sp1 faces the bottom surface.

Configuration of Second Gripping Member

The second gripping member 9 has an elongated shape extending in the longitudinal direction of the gripping portion 7. Further, the second gripping member 9 is pivotally supported to be rotatable with respect to the shaft 6 about a fulcrum P1 (FIGS. 1 and 2) on the proximal end side Ar2. In addition, the second gripping member 9 is pivotally supported to be rotatable with respect to the movable member 61 about a fulcrum P2 (FIGS. 1 and 2) on the proximal end side Ar2. That is, the second gripping member 9 rotates about the fulcrum P1 when the movable member 61 moves back and forth along the central axis Ax in response to the operation of the operation knob 51 performed by the operator. As a result, the second gripping member 9 is open and closed with respect to the first gripping member 8.

Here, a lower surface in FIG. 2 in the second gripping member 9 functions as the gripping surface 91 for gripping the target site together with the treatment surface 121. The gripping surface 91 can be formed of a flat surface orthogonal to the direction A1.

Incidentally, the first gripping member 8 (support member 10) can be fixed to the shaft 6 and the second gripping member 9 is pivotally supported with respect to the shaft 6, but the disclosure is not limited thereto. For example, a configuration may be adopted in which both the first and second gripping members 8 and 9 are pivotally supported with respect to the shaft 6 and rotate to open and close the first and second gripping members 8 and 9. In addition, for example, a configuration may be adopted in which the first gripping member 8 is pivotally supported with respect to the shaft 6, the second gripping member 9 is fixed to the shaft 6, and the first gripping member 8 is rotated to be open and closed with respect to the second gripping member 9.

Configurations of Control Apparatus and Foot Switch

The foot switch 4 is a part that is operated by the operator with a foot. Further, the treatment control by the control apparatus 3 is executed in response to the operation on the foot switch 4.

Incidentally, means for executing the treatment control is not limited to the foot switch 4, and a switch or the like operated by hand may be adopted.

The control apparatus 3 includes a central processing unit (CPU) and the like, and executes the treatment control for treatment of a target site by operating the treatment tool 2 according to a predetermined program.

Operation of Treatment System

Next, an operation of the treatment system 1 described above will be described.

The operator holds the treatment tool 2 by the hand and inserts a distal end portion (each part of the gripping portion 7 and the shaft 6) of the treatment tool 2 into an abdominal cavity after passing through an abdominal wall using, for example, a trocar. In addition, the operator operates the operation knob 51. Further, the operator grips the target site by the gripping portion 7. Thereafter, the operator operates the foot switch 4. Further, the control apparatus 3 executes the following treatment control.

The control apparatus 3 applies a voltage to the first and second connection portions 151 and 152 via the pair of lead wires C1. Here, the control apparatus 3 measures a resistance value of the conductive portion 15 (hereinafter referred to as a heater resistance) from a voltage value and a current value supplied to the conductive portion 15, for example, using a voltage drop method. In addition, the control apparatus 3 refers to resistance temperature characteristics measured in advance. Incidentally, the resistance temperature characteristic is a characteristic representing the relationship between the heater resistance and a temperature of the heat generating portion 153 (hereinafter referred to as a heater temperature). Further, the control apparatus 3 controls the heater resistance to a target resistance value corresponding to a target temperature in the resistance temperature characteristics while changing the electric power supplied to the conductive portion 15. As a result, the heater temperature is controlled to the target temperature. That is, the heat from the heat generating portion 153 controlled to the target temperature is transferred to the target site via the treatment member 12.

With the above treatment control, the target site is incised while coagulating.

Method of Manufacturing Treatment Tool

Next, a method of manufacturing the above-described treatment tool 2 will be described.

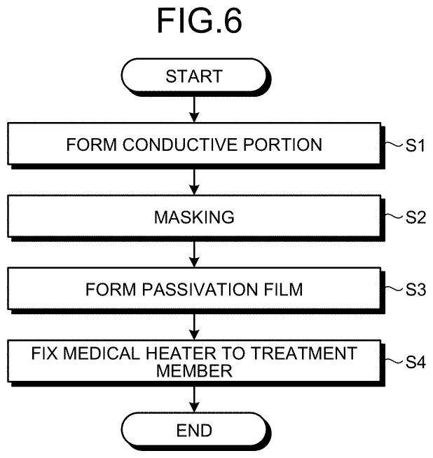

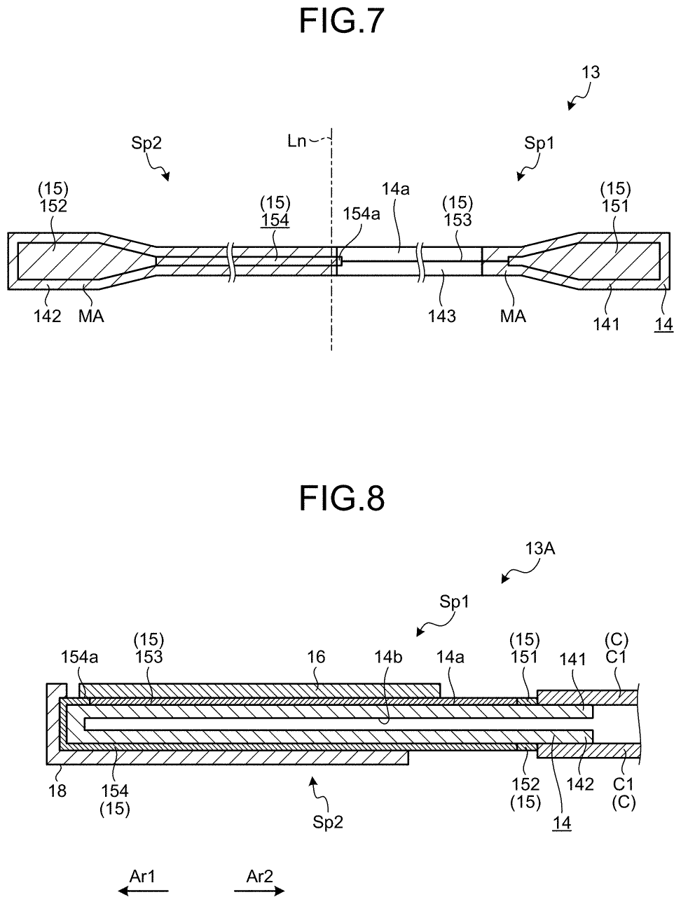

FIG. 6 is a flowchart illustrating the method of manufacturing the treatment tool 2. FIG. 7 is a view illustrating the method of manufacturing the treatment tool 2. Specifically, FIG. 7 is a view corresponding to FIG. 4.

First, an operator forms the conductive portion 15 on the first plate surface 14a of the substrate 14 by sputtering or the like (Step S1).

After Step S1, the operator masks a region other than a region where the passivation film 16 is to be provided using a tape or the like (Step S2). Incidentally, a masked region MA is represented by diagonal lines in FIG. 7 for convenience of the description.

After Step S2, the operator places the substrate 14 in an atmosphere of a gas containing fluorine and performs heating to a predetermined temperature to perform surface modification of the region other than the masked region MA on the surface of the conductive portion 15 (Step S3). As a result, the passivation film 16 made of nickel fluoride is formed in the region other than the masked region MA, that is, on a part of the surface of the heat generating portion 153 and the surface of the heat-generating-side end portion 154a. Thereafter, the operator removes the tape or the like used for masking in Step S2.

After Step S3, the operator folds the substrate 14 with the folding line Ln as the reference as illustrated in FIG. 5 in a state where the first plate surface 14a forms the outer surface. In addition, the operator fixes the medical heater 13 to the bottom surface of the recess 123 by the adhesive sheet 17 in a posture in which the folding line Ln is located on the distal end side Ar1 and in a state where the treatment-side region Sp1 faces the bottom surface (Step S4).

According to the exemplary embodiment described above, the following effects are achieved.

In the medical heater 13, the heat generating portion 153 can be made of the material containing nickel. In addition, a part of the surface of the heat generating portion 153 is covered with the passivation film 16 made of nickel fluoride.

Here, it is assumed a case where a part of the medical heater 13 is peeled off from the bottom surface of the recess 123 depending on the application of the treatment tool 2 so that a state where a part of the treatment-side region Sp1 on the first plate surface 14a is exposed in the recess 123 is formed. Even in this case, a part of the surface of the heat generating portion 153 is covered with the passivation film 16, and thus, it is possible to suppress corrosion or oxidation of the heat generating portion 153 and generation of rust in the heat generating portion 153 which cause a change in the resistance temperature characteristic measured in advance. That is, even when the treatment tool 2 is used for a long period of time, the heater temperature can be controlled to the target temperature by using the resistance temperature characteristic measured in advance.

In particular, the heat generating portion 153 is made of the material containing nickel. In addition, the passivation film 16 is made of nickel fluoride.

Therefore, if a part of the surface of the heat generating portion 153 is exposed to an atmosphere containing fluorine so that a predetermined heat is applied, the passivation film 16 is formed by surface modification of the heat generating portion 153. That is, a special device is not required to form the passivation film 16, and the manufacturing cost of the medical heater 13 can be reduced. In addition, since the passivation film 16 is formed by the surface modification of the heat generating portion 153, the passivation film 16 can be formed as a dense film, and a thickness dimension of the passivation film 16 can be made extremely small. Therefore, the passivation film 16 does not cause deterioration in thermal conductivity from the heat generating portion 153 to the treatment member 12. That is, the performance in treatment of the target site is not lowered.

In addition, the conductive portion 15 is provided on the first plate surface 14a in the state of being connected in series in an order of the first connection portion 151, the heat generating portion 153, the electric path portion 154, and the second connection portion 152 along the longitudinal direction of the substrate 14. Further, the substrate 14 is folded with the folding line Ln as the reference in the state where the first plate surface 14a forms the outer surface of the medical heater 13. Further, the medical heater 13 is fixed to the bottom surface of the recess 123 by the adhesive sheet 17 in the state where the treatment-side region Sp1 faces the bottom surface. That is, the substrate 14 having electrical insulation is present between the treatment-side region Sp1 in the conductive portion 15 and the back-surface-side region Sp2 in the conductive portion 15.

Therefore, it is possible to prevent a short circuit from occurring between the treatment-side region Sp1 in the conductive portion 15 and the back-surface-side region Sp2 in the conductive portion 15.

Meanwhile, for example, in a medical heater disclosed in US 2015/0327909 A1, first and second connection portions constituting a conductive portion are arranged side by side in a width direction of a substrate on a proximal end side of the substrate. In addition, a heat generating portion constituting the conductive portion has a substantially U-shape which extends from the proximal end side toward a distal end side and is folded at the distal end side to extend toward the proximal end side. Further, both ends of the heat generating portion are electrically connected to the first and second connection portions, respectively. That is, the conductive portion has two electric paths parallel to each other in the width direction of the substrate. In such a configuration, it is necessary to sufficiently separate the two electric paths in order to prevent the short circuit in the two electric paths. That is, a width dimension of the substrate becomes large.

On the other hand, in the medical heater 13 according to an exemplary embodiment, the conductive portion 15 is configured to extend along the longitudinal direction (left-right direction in FIG. 4) of the substrate 14. Further, when the substrate 14 is folded with the folding line Ln as the reference, the treatment-side region Sp1 in the conductive portion 15 and the back-surface-side region Sp2 in the conductive portion 15 are parallel to each other in the direction A1. That is, it is unnecessary to arrange the two electric paths in parallel in the width direction of the substrate 14 as described above, and the width dimension of the substrate 14 can be reduced.

In addition, the electric path portion 154 is provided so as to straddle the folding line Ln in the medical heater 13. That is, the electric path portion 154 is folded in the state where the substrate 14 is folded with the folding line Ln as the reference. Here, the electric path portion 154 is set to have a larger cross-sectional area than the heat generating portion 153. Therefore, the disconnection of the conductive portion 15 can be suppressed as compared with a case where the heat generating portion 153 is folded, and the durability of the conductive portion 15 can be sufficiently ensured.

In addition, the passivation film 16 covers not only the surface of the heat generating portion 153 but also the surface of the heat-generating-side end portion 154a of the electric path portion 154 in the medical heater 13. Here, the heat-generating-side end portion 154a is the portion connected to the heat generating portion 153, and thus, the temperature is likely to become high. That is, the corrosion or oxidation of the heat-generating-side end portion 154a and the generation of rust in the heat-generating-side end portion 154a are likely to occur depending on the application of the treatment tool 2.

Therefore, it is possible to suppress the corrosion or oxidation of the heat-generating-side end portion 154a and the generation of rust in the heat-generating-side end portion 154a, which cause the change in resistance temperature characteristic measured in advance, by covering the surface of the heat-generating-side end portion 154a with the passivation film 16. That is, even when the treatment tool 2 is used for a long period of time, the heater temperature can be controlled to the target temperature by using the resistance temperature characteristic measured in advance.

Next, another exemplary embodiment will be described.

In the following description, the same reference signs are given to the same configurations as those of the above-described embodiment, and a detailed description thereof will be omitted or simplified.

FIG. 8 is a view illustrating a medical heater 13A according to an exemplary embodiment. Specifically, FIG. 8 is a view corresponding to FIG. 5.

As illustrated in FIG. 8, the medical heater 13A according to an exemplary embodiment that is different from the medical heater 13 described in the above-described embodiment in terms that a cover member 18 is added.

The cover member 18 is provided on the first plate surface 14a of the substrate 14 so as to straddle the folding line Ln. Specifically, the cover member 18 extends from a position, which has a predetermined gap from the passivation film 16 toward the second connection portion 152, toward the second connection portion 152 to cover the surface of the electric path portion 154. That is, the cover member 18 covers a region of the electric path portion 154 other than the heat-generating-side end portion 154a.

As the cover member 18 described above, a material having electrical insulation, for example, a coverlay, a sealing material, a melt layer of polyimide, or the like can be exemplified.

According to the exemplary embodiment described above, not only the same effects as those in the above-described embodiment but also the following effects are obtained.

The medical heater 13A can be provided with the cover member 18.

Therefore, the cover member 18 can improve the watertightness of the back-surface-side region Sp2 in the conductive portion 15. In addition, since the cover member 18 has electrical insulation, it is possible to prevent the short circuit from occurring between the treatment-side region Sp1 in the conductive portion 15 and the back-surface-side region Sp2 in the conductive portion 15 even when a liquid enters the recess 111.

In addition, the cover member 18 covers the region of the electric path portion 154 other than the heat-generating-side end portion 154a. That is, the cover member 18 is provided at a position avoiding the heat-generating-side end portion 154a, which is likely to become a high temperature, and thus, the cover member 18 does not become a high temperature, and it is possible to avoid peeling of the cover member 18 from the first plate surface 14a.

### Other Embodiments

The modes for carrying out the disclosure have been described hereinbefore. However, the disclosure is not limited only to the exemplary embodiments described above.

Although the substrate 14 constituting the medical heater 13 or 13A according to the disclosure is made of the resin material such as polyimide in the above-described embodiments, the disclosure is not limited thereto, and a ceramic substrate may be adopted. When the ceramic substrate is adopted, the ceramic substrate may be provided with the treatment surface that comes into contact with the target site.

Although the configuration in which thermal energy is applied to the target site has been adopted in the above-described embodiments, the disclosure is not limited thereto, and a configuration in which high frequency energy or ultrasonic energy is applied in addition to the thermal energy may be adopted. Incidentally, “applying the high frequency energy to a target site” means causing a high frequency current to flow to the target site. In addition, “applying the ultrasonic energy to a target site” means applying an ultrasonic vibration to the target site.

Although the medical heater 13 or 13A according to the disclosure is provided only on the first gripping member 8 in the above-described embodiments, but the disclosure is not limited thereto, and the medical heater 13 or 13A according to the disclosure may be provided on both the first and second gripping members 8 and 9.

With a medical heater, a treatment tool, and a method of manufacturing a treatment tool according to the disclosure, it is possible to suppress a change of a resistance temperature characteristic in a heat generating portion at a low manufacturing cost without lowering performance in treatment of a target site.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical heater comprising:
a heat generating portion that is made of a material that includes nickel, the heat generating portion being configured to generate heat when energized; and
a passivation film that includes nickel fluoride, the passivation film being configured to cover at least a part of a surface of the heat generating portion.

2. The medical heater according to claim 1, wherein
the heat generating portion is made of a material that includes one of: stainless steel, nickel, or a nickel alloy.

3. The medical heater according to claim 1, further comprising:
a substrate that includes:
electrical insulation;
a first plate surface; and
a second plate surface forming front and back surfaces of the substrate;
a first connection portion electrically connected to a first wiring member and a second connection portion electrically connected to a second wiring member; and
an electric path portion configured to provide an energization path to the heat generating portion,
wherein:
the heat generating portion, the first connection portion, the second connection portion, and the electric path portion are provided on the first plate surface and are connected in series along a longitudinal direction of the substrate in an order of the first connection portion, the heat generating portion, the electric path portion, and the second connection portion,
the heat generating portion has a higher resistance value than the first connection portion, the second connection portion, and the electric path portion, and
the substrate is made of a flexible material, and is folded such that the substrate includes a folding line orthogonal to the longitudinal direction of the substrate when the first plate surface forms an outer surface of the medical heater.

4. The medical heater according to claim 3, wherein
the electric path portion is configured to straddle the folding line and is made of a material that includes nickel, and
the passivation film is configured to cover at least a part of a surface of the heat generating portion and a surface of a heat-generating-side end portion of the electric path portion, the heat-generating-side end portion being connected to the heat generating portion.

5. The medical heater according to claim 4, further comprising a cover member that is made of a material including electrical insulation, the cover member being configured to cover a region of the electric path portion other than the heat-generating-side end portion.

6. A treatment tool comprising:
a treatment member having a treatment surface for treating a living tissue and an installation surface forming front and back surfaces of the treatment member with the treatment surface; and
a medical heater configured to heat the treatment member,
wherein:
the medical heater includes:
a substrate that is made of a material having electrical insulation and has a first plate surface and a second plate surface forming front and back surfaces of the substrate;
a heat generating portion that is made of a material that includes nickel and generates heat when energized;
a passivation film that is made of nickel fluoride, the passivation film being configured to cover at least a part of a surface of the heat generating portion;
a first connection portion electrically connected to a first wiring member and a second connection portion electrically connected to a second wiring member; and
an electric path portion configured to provide an energization path to the heat generating portion,
the heat generating portion, the first connection portion, the second connection portion, and the electric path portion are provided on the first plate surface and connected in series along a longitudinal direction of the substrate in an order of the first connection portion, the heat generating portion, the electric path portion, and the second connection portion,
the heat generating portion has a first resistance value that is higher than a second resistance value of the first connection portion, a third resistance value of the second connection portion, and a fourth resistance value of the electric path portion, and
the substrate is made of a flexible material and includes a folding line orthogonal to the longitudinal direction of the substrate as a reference when the first plate surface forms an outer surface of the medical heater, and
the substrate is installed such that the heat generating portion faces the installation surface.

7. The treatment tool according to claim 6, further comprising an adhesive sheet that is made of a material having electrical insulation, the adhesive sheet being configured to adhere the first plate surface to the installation surface.

8. A method of manufacturing a treatment tool, the method comprising:
forming a heat generating portion, the heat generating portion being made of a material that includes nickel and generates heat when energized;
electrically connecting a first connection portion to a first wiring member and a second connection portion to a second wiring member; and
forming an electric path portion to provide an energization path to the heat generating portion on a first plate surface of a substrate when the first connection portion, the heat generating portion, the electric path portion, and the second connection portion are sequentially connected in series along a longitudinal direction of the substrate; and
performing surface modification on at least a part of a surface of the heat generating portion in an atmosphere of a gas containing fluorine to form a passivation film made of nickel fluoride on at least the part of the surface of the heat generating portion.

9. The method of manufacturing a treatment tool according to claim 8, further comprising:
folding the substrate to form a folding line orthogonal to the longitudinal direction of the substrate as a reference when the first plate surface forms an outer surface, and the substrate is installed on a treatment member when the heat generating portion faces an installation surface of the treatment member configured to treat a living tissue.
